(19) United States
(12) Patent Application Publication
(10) Pub. No.: US 2008/0274213 A1
(43) Pub. Date: Nov. 6, 2008

(54) Drug for Ameliorating Male Climacteric Disorders

(75) Inventors: Setsuko Sekita, Kagawa (JP); Masanori Kuroyanagi, Hiroshima (JP); Kayo Yasuda, Hiroshima (JP); Ikuko Mizuno, Hiroshima (JP); Mitsuyasu Ushijima, Hiroshima (JP); Minoru Hayama, Hiroshima (JP); Makoto Ichikawa, Hiroshima (JP); Masanori Sumihiro, Hiroshima (JP)

Correspondence Address: OBLON, SPIVAK, MCCLELLAND MAIER & NEUSTADT, P.C., 1940 DUKE STREET, ALEXANDRIA, VA, 22314, US

(73) Assignee: WAKUNAGA PHARMACEUTICAL CO., LTD., Osaka-shi, Osaka (JP)

(21) Appl. No.: 11/597,924

(22) PCT Filed: May 31, 2005
(86) PCT No.: PCT/JP2005/009937

§ 371 Date: Nov. 29, 2006

(30) Foreign Application Priority Data
May 31, 2004 (JP) 2004-160445

(51) Int. Cl. A61K 36/00; A61K 31/70; A61P 15/12

(52) U.S. Cl. 424/725; 514/25

(57) ABSTRACT

It is intended to provide a highly safe pharmaceutical drug or food that inhibits reduction in blood testosterone level and prevents or ameliorates symptoms and so on associated with male climacteric disorders. The present invention provides a drug for inhibiting reduction in blood testosterone level and a drug for ameliorating male climacteric disorders comprising a plant belonging to the genus Codonopsis or an extract thereof.

### TECHNICAL FIELD

The present invention relates to a pharmaceutical drug having inhibitory effects on the reduction in blood testosterone level and ameliorating effects on male climacteric disorders, and also to food having the same effects.

### BACKGROUND ART

Testosterone is said to have influential roles in the development of the male reproductive organ, the development of bone structure and muscles, the enhancement of sexual desire and instinct, and even the enhancement of brain and mental vitality. Also, the concentration of testosterone in blood is known to decrease when affected by a stress. A decrease of blood testosterone level could lead to diseases such as male climacteric disorders and delayed puberty.

Male climacteric disorders are assumed to be strongly implicated in the manifestation of diverse symptoms (e.g., dejection, depression, irritability, anxiety, nervousness, loss of spirits, fatigue, arthritis, myositis, muscular weakness, sudation, hot flash, sleep disorder, deterioration of memory, reduced concentration, physical exhaustion, low sexual desire, erectile dysfunction, and decreased awareness of ejaculation). Among the methods already reported as detecting climacteric disorders are a blood testosterone concentration measurement by a blood test and a method by a questionnaire survey. The onset of a male climacteric disorder is observed largely in males aged 40 to 50, but in some cases, its symptoms can be observed as early as their twenties, or even as late as their sixties. It is pointed out that the onset of male climacteric disorders is deeply associated with various stresses, which most of the male workers are experiencing at the peak of his career and at home. In recent years, the number of middle-aged and older male patients suspected of having climacteric disorders is increasing.

Hormone replacement therapies using androgen preparations containing testosterone are prevailing as a clinical way to reduce blood testosterone levels and cure male climacteric disorders, and its effectiveness on various symptoms has been reported in practice (see e.g., Non-Patent Document 1). However, individuals suited for this therapy are limited to male patients who have apparently shown low testosterone levels in blood tests and are suffering from no prostate disease. This is because the therapy has the possibility of bringing about the manifestation of prostatic hypertrophy or prostatic cancer as a side effect. Thus there has been urgent demanded for the development of a pharmaceutical drug or healthy food with few side effects composed of safe materials, which can be expected to prevent and mitigate reduction in blood testosterone level and symptoms associated with male climacteric disorders.

Codonopsis lanceolata has long been used as a folk medicine for anti-inflammation, expectoration, nutritional fortification, invigoration and soon, and this plant has also been used as a herbal food, particularly in South Korean recipe. Moreover, this plant was reported to be useful as an ingredient of powdered food or beverage (see e.g., Patent Documents 1 to 2). Even more, this plant has spermatogenesis-promoting effect and impaired sexual behavior-ameliorating effect, according to a study made on the pharmacological effect and components thereof. (see e.g., Non-Patent Document 2).

Nevertheless, there has been no report focused on the inhibitory effect on reduction in blood testosterone level and ameliorating effect on male climacteric disorders, which might be brought about by Codonopsis lanceolata and its components.

[Patent Document 1] Japanese Patent Laid-Open No. 2001-299273

[Patent Document 2] Japanese Patent Laid-Open No. 2002-218941

[Non-Patent Document 1] Naoki Ito, Shinichi Hisasue, and Taiji Tsukamoto, Male Hormone Replacement Therapy for Male climacteric disorders, Geriat. Med. 42 (9): 1151-1156, 2004

[Non-Patent Document 2] Years Heisei 10-12 (1998-2000) Proceedings of Basic Research of Health Science Research Including Drug Innovation, Project V, Research Regarding Development of Healthy Life Extension/Preventive Drugs, 86-99, 2001

### DISCLOSURE OF THE INVENTION

### Problems that the Invention is to Solve

An object of the present invention is to provide a highly safe pharmaceutical drug or food that inhibits reduction in blood testosterone level and prevents or ameliorates symptoms and so on associated with male climacteric disorders.

### Means for Solving the Problems

The present inventors have searched a variety of highly safe natural materials and have consequently completed the present invention by finding out that a plant belonging to the genus Codonopsis or an extract thereof and particular saponin and phenyl propanoids contained in the plant remarkably inhibit reduction in blood testosterone level and exert excellent ameliorating effect on the symptoms of male climacteric disorders.

Namely, the present invention relates to a drug for inhibiting reduction in blood testosterone level comprising a plant belonging to the genus Codonopsis or an extract thereof.

The present invention also relates to a drug for ameliorating male climacteric disorders comprising a plant belonging to the genus Codonopsis or an extract thereof.

The present invention also relates to a food comprising a plant belonging to the genus Codonopsis or an extract thereof and comprising an indication stating that the food is used for inhibiting reduction in blood testosterone level or preventing, treating, ameliorating, or alleviating male climacteric disorders.

The present invention also relates to a drug for inhibiting reduction in blood testosterone level comprising one kind or two or more kinds of members selected from tangshenosides, lancemaside-A, and syringin.

The present invention also relates to a drug for ameliorating male climacteric disorders comprising one kind or two or more kinds of members selected from tangshenosides, lancemaside-A, and syringin.

The present invention also relates to a pharmaceutical drug comprising one kind or two or more kinds of members selected from tangshenosides, lancemaside-A, and syringin.

The present invention also relates to a food comprising one kind or two or more kinds of members selected from tangshenosides, lancemaside-A, and syringin.

The present invention also relates to use of a plant belonging to the genus Codonopsis or an extract thereof for producing a drug for inhibiting reduction in blood testosterone level.

The present invention also relates to use of a plant belonging to the genus Codonopsis or an extract thereof for producing a drug for ameliorating male climacteric disorders.

The present invention also relates to use of tangshenosides, lancemaside-A, and syringin for producing a drug for inhibiting reduction in blood testosterone level.

The present invention also relates to use of tangshenosides, lancemaside-A, and syringin for producing a drug for ameliorating male climacteric disorders.

The present invention also relates to a method for inhibiting reduction in blood testosterone level, characterized by administering or ingesting a plant belonging to the genus Codonopsis or an extract thereof.

The present invention also relates to a method for ameliorating male climacteric disorders, characterized by administering or ingesting a plant belonging to the genus Codonopsis or an extract thereof.

The present invention also relates to a method for inhibiting reduction in blood testosterone level, characterized by administering or ingesting one kind or two or more kinds of members selected from tangshenosides, lancemaside-A, and syringin.

The present invention also relates to a method for ameliorating male climacteric disorders, characterized by administering or ingesting one kind or two or more kinds of members selected from tangshenosides, lancemaside-A, and syringin.

### ADVANTAGE OF THE INVENTION

The drug for inhibiting reduction in blood testosterone level and the drug for ameliorating male climacteric disorders of the present invention can inhibit reduction in blood testosterone level and prevent, treat, ameliorate, and alleviate symptoms associated with male climacteric disorders and delayed puberty, without side effects such as the manifestation of prostatic hypertrophy and prostatic cancer.

### BRIEF DESCRIPTION OF THE DRAWING

FIG. 1 is a diagram showing changes in the blood testosterone concentrations of restraint stress-loaded mice in groups receiving with the present invention and control groups (mean ±standard deviation).

### BEST MODE FOR CARRYING OUT THE INVENTION

In the present invention, the inhibition of reduction in blood testosterone level means the inhibition of reduction in blood testosterone concentration to below normal levels due to stress, aging, and so on. Thus, a drug for inhibiting reduction in blood testosterone level is useful for preventing and ameliorating diseases and symptoms attributed to reduction in blood testosterone level, for example, male climacteric disorders and delayed puberty.

In the present invention, the amelioration of male climacteric disorders means the prevention, treatment, amelioration, and alleviation of symptoms caused by male climacteric disorders, for example, dejection, depression, irritability, anxiety, nervousness, loss of spirits, fatigue, arthritis, myositis, muscular weakness, sudation, hot flash, sleep disorder, deterioration of memory, reduced concentration, physical exhaustion, low sexual desire, erectile dysfunction, decreased awareness of ejaculation, and aging. The male climacteric disorder encompasses all disorders that exhibit these symptoms regardless of ages.

In the present invention, examples of a plant belonging to the genus Codonopsis include Codonopsis lanceolata Trautv., C. pilosula Nannf., and C. sylvestris. Among them, the C. lanceolata Trautv. is preferable.

A whole or partial element constituting a plant body can be used as the plant belonging to the genus Codonopsis. For example, roots, rhizomes, leaves, stems, flowers, fruits, seeds, and buds can be used. The roots or rhizomes are preferably used.

An extract of the plant belonging to the genus Codonopsis includes a variety of solvent extracted solutions obtained by extracting the plant belonging to the genus Codonopsis at room temperature or under heating or by use of an extraction apparatus such as a Soxhlet extractor, diluted solutions thereof, concentrated solutions thereof, extracts thereof, and dried products obtained by drying them.

Both polar and nonpolar solvents can be used as extraction solvents for obtaining the extract of the present invention, and a mixture thereof can also be used. Examples thereof include: water; alcohols such as methanol, ethanol, 1-propanol, 2-propanol, and 1-butanol; linear and cyclic ethers such as 1,4-dioxane, tetrahydrofuran, and diethyl ether; ketones such as acetone and methyl ethyl ketone; nitrites such as acetonitrile; esters such as methyl acetate and ethyl acetate; polyethers such as polyethylene glycol; halogenated hydrocarbons such as dichloromethane, chloroform, and carbon tetrachloride; hydrocarbons such as hexane, cyclohexane, and petroleum ether; aromatic hydrocarbons such as benzene and toluene; pyridines; supercritical carbon dioxide; and fats and oils, wax, and other oils. Among them, the polar solvents, particularly water, ethanol, and a mixed solution thereof are preferably used.

The extraction can be performed by a routine method, though differing depending on solvents used. For example, the plant body or dried product thereof may be pulverized, crushed, or cut, then supplemented with a polar solvent, and left at 0° C. to 100° C., preferably 70° C. to 100° C., for 5 minutes to 24 hours, preferably 5 minutes to 1 hour.

The extract can be used directly or after being subjected to dilution, the removal of insoluble matter by appropriate procedures such as filtration or centrifugation, and the removal of the solvent, and then concentrated or freeze-dried, and, if necessary, prepared into a powder or paste.

Alternatively, the extract can be used by removing inactive impurities therefrom by use of purification techniques such as liquid-liquid distribution techniques (e.g., washing with a nonpolar solvent such as ethyl acetate, diethyl ether, and hexane and extraction with water) and a variety of chromatography approaches. In the present invention, such purified products are preferably used. They may also be used, if necessary, after being subjected to treatment such as deodorization and decolorization by a method known in the art.

In the present invention, tangshenosides, lancemaside-A, and syringin have chemical structures as shown below. The tangshenosides include tangshenoside I and tangshenoside II.

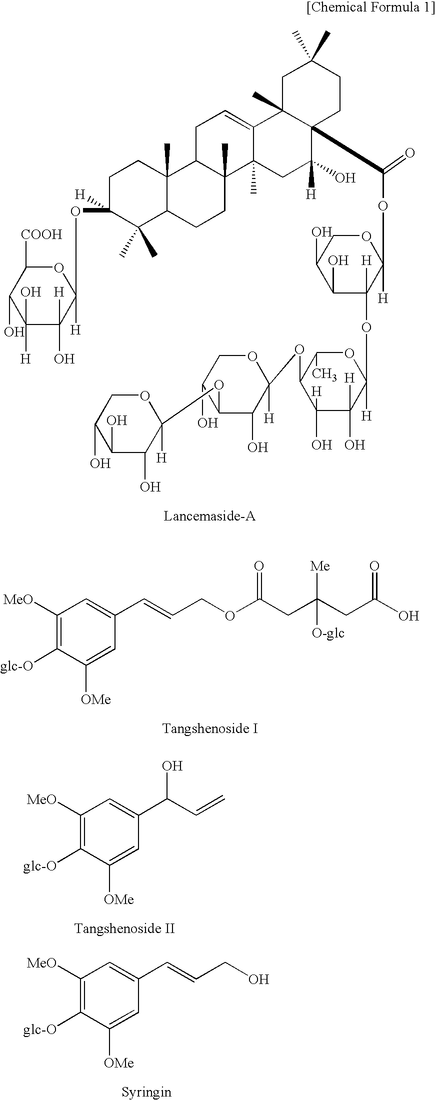

All of them are components contained in the plant belonging to the genus Codonopsis and can be obtained by subjecting Codonopsis lanceolata to extraction with alcohols such as methanol and applying the resulting extract to separation and purification by a variety of column chromatography approaches and subsequent high performance liquid chromatography, as shown in Examples 2 and 3 below. In this context, lancemaside-A is a novel compound isolated de novo.

In the present invention, the compounds described above are not limited to this method and may be those extracted from other natural products or synthesized chemically.

The plant belonging to the genus Codonopsis or extract thereof, tangshenosides, and lancemaside-A of the present invention (hereinafter, also referred to as the Codonopsis plant and so on”) possess excellent inhibitory effect on reduction in blood testosterone level and ameliorating effect on male climacteric disorders and also have high safety, as shown in Examples below. Therefore, they can be made into a drug for inhibiting reduction in blood testosterone level and a drug for ameliorating male climacteric disorders available as foods and pharmaceutical drugs.

The Codonopsis plant and so on of the present invention, when used as a food, can be made into foods based on the concept of the prevention, amelioration, or alleviation of symptoms associated with male climacteric disorders (e.g., dejection, depression, irritability, anxiety, nervousness, loss of spirits, fatigue, arthritis, myositis, muscular weakness, sudation, hot flash, sleep disorder, deterioration of memory, reduced concentration, physical exhaustion, low sexual desire, erectile dysfunction, decreased awareness of ejaculation, and aging), for example, foods for invalids and foods for specified health use comprising an indication on products, packages, catalogs, files, and so on, stating so.

Possible food forms thereof are all forms such as solid foods, semi-fluid foods (e.g., cream or jam forms), gel foods, drinks, and tea leaves. Examples thereof include powder, capsule, granule, tablet, drinkable preparation, and tea bag forms. Such foods and drinks can be processed according to a routine method.

The food described above can be supplemented with medicinal plants conventionally used for the amelioration and so on of male climacteric disorders, for example, plants having antidepressive effect, plants having antianxiety effect, plants having inhibitory effect on reduction in blood DHEA-S (dehydroepiandrosterone sulfate) level, or extracts thereof. These plants may have any two or more effects of antidepressive effect, antianxiety effect, and inhibitory effect on reduction in blood DHEA-S level in themselves.

Examples of the plants having antidepressive effect include Korean ginseng, Siberian ginseng, Saint John's wort, damiana, ginkgo leaves, valerian, green tea, passionflower, hop, chamomile, skullcap, jujube, lotus seeds, kavakava, pomegranate, orange flowers, lemon verbena, linden, marjoram, passionflower, lemon balm, jasmine, lavender, mint, saffron, cola, corktree, Japanese white-bark magnolia, cicely, perilla, and rafuma (Apocynum venetum), with the rafuma preferred.

Examples of the plants having antianxiety effect include kawa, rose, kava, bacopa, clary sage, geranium, valerian, chamomile, Korean ginseng, Siberian ginseng, lavender, ginkgo leaves, lemon verbena, saffron, passionflower, and kavakava.

Examples of the plants having inhibitory effect on reduction in blood DHEA-S level include yam, Korean ginseng, guarana, Magnolia Vine, damiana, Gotu Kola, and sophon, with the sophon preferred.

The Codonopsis plant and so on of the present invention, when used as a pharmaceutical drug, may be made into a pharmaceutical composition by adding a pharmaceutically acceptable carrier to the plant belonging to the genus Codonopsis or extract thereof, tangshenosides, lancemaside-A, or syringin.

The mode of administration of the pharmaceutical drug of the present invention is not particularly limited and can include ordinary administration routes such as oral administration, rectal administration, transdermal administration, and administration by injection, with the oral administration preferred.

The pharmaceutical drug can be formulated into a solid preparation, liquid preparation, or the like, appropriate to administration route with a pharmaceutically acceptable carrier.

The solid preparation for oral administration includes capsules, tablets, pills, troches, powders, and granules. The solid preparation can generally be prepared by mixing the composition described in the present specification with at least one kind of additive (e.g., crystalline cellulose, lactose, or starch). This preparation may also be prepared by using a lubricant such as magnesium stearate, in addition to the additive. A buffer may further be used in the capsule, tablet, and pill. The tablet and pill can be enteric-coated.

The liquid preparation for oral administration includes pharmaceutically acceptable emulsions, solutions, suspensions, syrups, and elixirs containing inactive diluents usually used for preparing liquid preparations, for example, water. The liquid preparation can be prepared by adding an additive to the plant body and/or extract of the present invention and further mixing it with an auxiliary, for example, a lubricant, emulsifier, suspending agent, seasoning, or flavoring.

The pharmaceutical drug can be supplemented with the above-described medicinal plants conventionally used for the amelioration and so on of male climacteric disorders or with drugs used for the treatment and so on of male climacteric disorders, for example, drugs having antidepressive effect, drugs having antianxiety effect, and drugs having inhibitory effect on reduction in blood DHEA-S level and can thereby exert inhibitory effect on reduction in blood testosterone level and ameliorating effect on male climacteric disorders more effectively.

Examples of the drugs having antidepressive effect include tricyclic antidepressants such as amitriptyline hydrochloride, tetracyclic antidepressants such as maprotiline hydrochloride, SSRI such as fluvoxamine maleate, SNRI such as milnacipran hydrochloride, MAO inhibitors such as safrazine hydrochloride, trazodone hydrochloride, and sulpiride.

Examples of the drugs having antianxiety effect include benzodiazepine derivatives such as alprazolam, etizolam, oxazolam, and cloxazolam, and tandospirone.

Examples of the drugs having inhibitory effect on reduction in blood DHEA-S level include DHEA (dehydroepiandrosterone sulfate).

Examples of the preferable embodiment of the present invention include a composition obtained by adding, for example, rafuma having antidepressive effect and sophon having inhibitory effect on reduction in blood DHEA-S level and further an excipient or carrier generally used in pharmaceutical drugs or foods to an extract obtained by a method wherein Codonopsis lanceolata roots are heated in hot water for 30 minutes to 120 minutes, then subjected to the removal of insoluble components and the concentration of solvents, and dried.

The dose of the Codonopsis plant and so on of the present invention used as a pharmaceutical drug differs depending on administration methods and the purpose of usage. The dose of the plant belonging to the genus Codonopsis or extract thereof prepared into a solid preparation is usually 0.01 to 5 g per dose and 0.01 to 15 g per day, preferably 0.1 to 1 g per dose and 0.1 to 3 g per day, more preferably 0.5 to 2 g per day, in terms of the original plant quantity. The dose of the lancemaside-A, tangshenosides, or syringin prepared into a solid preparation is usually 0.01 to 0.5 g per dose and 0.01 to 1.5 g per day, preferably 0.1 to 1 g per dose and 0.1 to 0.3 g per day, more preferably 0.1 to 0.2 g per day.

Alternatively, the dose of the plant belonging to the genus Codonopsis or extract thereof prepared into a liquid preparation is 1 to 50 mL per dose with one to three doses per day as 0.02 to 10 w/v % solution in terms of the original plant quantity. The dose of the lancemaside-A, tangshenosides, or syringin prepared into a liquid preparation is 1 to 50 mL per dose with one to three doses per day as 0.01 to 0.5 w/v % solution.

Hereinafter, the present invention will be described with reference to Examples. However, the present invention is not intended to be limited to them.

### EXAMPLES

### Example 1

### Preparation of Codonopsis Lanceolata Extract

Dried Codonopsis lanceolata roots (1.4 kg) were supplemented with 13 L of purified water and heated in hot water at 90° C. or higher for 1 hour, followed by filtration of insoluble components. The obtained hot water-extracted solution was freeze-dried to obtain 476 g of Codonopsis lanceolata extract.

### Example 2

### Extraction of Tangshenosides and Syringin

The Codonopsis lanceolata extract (476 g) obtained in Example 1 was passed through a porous polystyrene resin (DIAION HP-20), then washed with water, and eluted with methanol. This methanol-eluted fraction was separated by silica gel chromatography with a mixed solution of chloroform and methanol as an elution solvent to obtain Fraction 1 (which contained tangshenoside II and syringin) and Fraction 2 (which contained tangshenoside I). The Fraction 1 was separated by high performance liquid chromatography (Fractionation Condition 1) and a separated fraction was concentrated to obtain tangshenoside II (50 mg) and syringin (70 mg). The Fraction 2 was separated by high performance liquid chromatography (Fractionation Condition 2) and a separated fraction was concentrated to obtain tangshenoside I (24 mg).

Fractionation Condition 1 (fractionation condition of tangshenoside II and syringin by high performance liquid chromatography)

Mobile phase: 25% acetonitrile

Flow rate: 8 mL/min

Detection wavelength: 210 nm

Column: YMC-Pack ODS-AQ (20 mm in internal diameter, 250 mm in length, and 5 μm in particle size, manufactured by YMC Co., Ltd.)

Column temperature: room temperature

Fractionation Condition 2 (fractionation condition of tangshenoside I by high performance liquid chromatography)

Mobile phase: 18% acetonitrile

Flow rate: 8 mL/min

Detection wavelength: 210 nm

Column: Mightysil RP-18 GP (20 mm in internal diameter, 250 mm in length, and 5 μm in particle size, manufactured by Kanto Chemical Co., Inc.)

Column temperature: room temperature

### Example 3

### Extraction of Lancemaside-A

Dried Codonopsis lanceolata roots (1 kg) were supplemented with 15 L of purified water and heated in hot water at 90° C. or higher for 1 hour, followed by filtration of insoluble components. The obtained hot water-extracted solution was dried to obtain 0.4 kg of Codonopsis lanceolata extract. The Codonopsis lanceolata extract (0.4 kg) was passed through a porous polystyrene resin (DIAION HP-20), then washed with water and 30% methanol, and eluted with methanol. This methanol-eluted fraction was separated by silica gel chromatography with a mixed solution of chloroform, methanol, and water as an elution solvent. A lancemaside-A-containing fraction was separated by high performance liquid chromatography (Fractionation Condition 3). The lancemaside-A-containing eluted solution was passed through the DIAION HP-20 column, then washed with water, and eluted with methanol, followed by concentration and freeze-drying to obtain lancemaside-A (110 mg). The physical properties of lancemaside-A are shown below.

Fractionation Condition 3 (fractionation condition of lancemaside-A by high performance liquid chromatography)

Mobile phase: 0.1% trifluoroacetic acid: acetonitrile (72:28) Flow rate: 9.99 mL/min

Detection wavelength: 210 nm

Column: TSK gel ODS-80TS (20 mm in internal diameter, mm in length, and 5 μm in particle size, manufactured by Tosoh Corp.)

Column temperature: 25° C.

### Physical Properties

1) Nature: white powder
2) MS: electrospray ionization method

Cation: m/z 1213, [M+Na]⁺, m/z 1229, [M+K]⁺, Anion: m/z 1189, [M-H]⁻

3) ¹³C-NMR (pyridine-d₅)

TABLE 1

| | |
|---|---|
| carbon | ppm |

Aglycone

| | |
|---|---|
| 1 | 38.8 |
| 2 | 26.7 |
| 3 | 89.1 |
| 4 | 39.6 |
| 5 | 55.9 |
| 6 | 18.5 |
| 7 | 33.5 |
| 8 | 40.0 |
| 9 | 47.0 |
| 10 | 37.0 |
| 11 | 23.8 |
| 12 | 122.7 |
| 13 | 144.4 |
| 14 | 42.1 |
| 15 | 36.2 |
| 16 | 74.1 |
| 17 | 49.6 |
| 18 | 41.3 |
| 19 | 47.0 |
| 20 | 31.0 |
| 21 | 36.0 |
| 22 | 32.2 |
| 23 | 28.2 |
| 24 | 17.0 |
| 25 | 15.7 |
| 26 | 17.6 |
| 27 | 27.2 |
| 28 | 176.0 |
| 29 | 33.3 |
| 30 | 24.8 |

Sugar-3
GlcU

| | |
|---|---|
| 1 | 107.3 |
| 2 | 75.4 |
| 3 | 78.2 |
| 4 | 73.5 |
| 5 | 77.9 |
| 6 | 172.9 |

Sugar-28
Ara

| | |
|---|---|
| 1 | 93.4 |
| 2 | 75.2 |
| 3 | 69.5 |
| 4 | 65.9 |
| 5 | 62.8 |

Rha

| | |
|---|---|
| 1 | 101.0 |
| 2 | 71.9 |
| 3 | 72.7 |
| 4 | 83.4 |
| 5 | 68.5 |
| 6 | 18.4 |

Xyl(inner)

| | |
|---|---|
| 1 | 106.2 |
| 2 | 75.0 |
| 3 | 87.1 |
| 4 | 69.0 |
| 5 | 66.9 |

Xyl′(terminal)

| | |
|---|---|
| 1 | 106.1 |
| 2 | 75.6 |
| 3 | 78.2 |
| 4 | 71.0 |
| 5 | 67.4 |

### Example 4

### Test on Inhibition of Reduction in Blood Testosterone Concentration

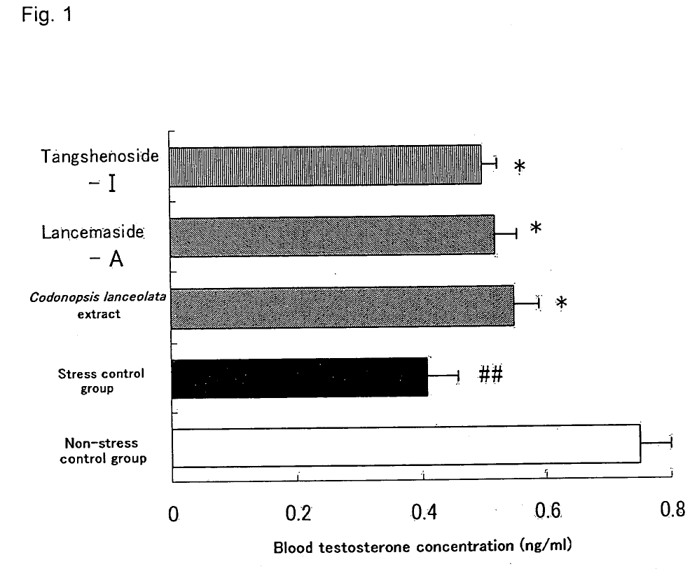

The Codonopsis lanceolata extract obtained in Example 1 was used to investigate inhibitory effect on reduction in blood testosterone concentration caused by aging and stress according to the following procedures: seven-month-old ddY male mice (10 mice per group) were orally given 1 g/kg of the test substance once a day over 2 weeks. On the last administration day, the mice were loaded with restraint stress after 1 hour of test substance administration. The restraint stress loading was performed by wrapping soft wire nets around the mouse bodies and allowing the mice to abstain from food and drink for 16 hours (17:00 to 9:00). Immediately after the termination of the loading, their blood testosterone concentrations were measured. A significant difference test used was the Student's t-test. The result is shown in FIG. 1. The blood testosterone concentration of the stress-loaded group was reduced as compared with a stress-unloaded group, whereas obvious inhibitory effect on reduction in blood testosterone concentration was observed in the Codonopsis lanceolata extract-administered group. This result showed significant difference with a significance level of 5% as compared with the stress-loaded group. Likewise, the inhibitory effect was also observed in lancemaside-A and tangshenoside I.

### Example 5

### Safety Study

To verify the safety of the Codonopsis lanceolata extract of the present invention obtained in Example 1, five-week-old ddY mice (four female and four male mice per group) were orally given a single dose (30 mL/kg as the amount of an administered solution) of 10 g/kg or 20 g/kg of the Codonopsis lanceolata extract. For subsequent 6 days, changes in body weight and general states were observed. On the next day and 6th day after the administration, two female and two male mice in each group were subjected to autopsy, and their thoracoabdominal organs were visually observed. No death was observed by the administration of the Codonopsis lanceolata extract. Moreover, all of the general states, changes in body weight, and autopsy reports were free of abnormal findings.

### Example 6

### Study on Amelioration of Male Climacteric Disorders

Three males in their forties (the age manifesting male climacteric disorders) took 0.7 g of a test substance (0.5 g of Codonopsis lanceolata extract (1.47 g in terms of the original plant quantity) and 0.2 g of excipients such as dextrin and starch) twice a day after breakfast and dinner for 14 days. Conditions after the administration were measured. As a result, all of these three males clinically seldom had morning erection serving as one of criterion symptoms of male climacteric disorders before the administration and however, had morning erection from three to four days after the initiation of the test substance administration. The morning erection was confirmed for a period until the termination of the administration.

### Example 7

### Formulation Example of Preparation

### (1) Granule

TABLE 2

| Component name | Formulation amount (mg/day) |
|---|---|
| Codonopsis lanceolata | 500 |
| extract | (1.47 g in terms of the |
| | original plant quantity) |
| Sophon powder | 250 |
| Rafuma extract powder | 75 |
| Dextrin | 150 |
| Crystalline cellulose | 500 |
| Lactose | 500 |
| Silicon dioxide | 25 |
| Total | 2000 |

(2) Liquid preparation

TABLE 3

| Component name | Formulation amount (mg/day) |
|---|---|
| Codonopsis lanceolata | 1000 |
| extract | (2.94 g in terms of the |
| | original plant quantity) |
| Siberian ginseng | 500 |
| Korean ginseng | 100 |
| Trehalose | 3000 |
| Fructose | 2000 |
| Hydrogenated castor oil | 250 |
| Sodium benzoate | 150 |
| Citric acid | Proper quantity |
| Water | Proper quantity |
| Total | 50 mL |

1. A drug for inhibiting reduction in blood testosterone level comprising a plant belonging to the genus Codonopsis or an extract thereof.

2. A drug for ameliorating male climacteric disorders comprising a plant belonging to the genus Codonopsis or an extract thereof.

3. A food comprising a plant belonging to the genus Codonopsis or an extract thereof and comprising an indication stating that the food is used for inhibiting reduction in blood testosterone level or preventing, treating, ameliorating, or alleviating male climacteric disorders.

4. The food according to claim 3, further comprising any one kind or two or more kinds of plants or extract(s) thereof selected from plants having antidepressive effect, plants having antianxiety effect, and plants having inhibitory effect on reduction in blood DHEA-S level.

5. A drug for inhibiting reduction in blood testosterone level comprising one kind or two or more kinds of members selected from tangshenosides, lancemaside-A, and syringin.

6. A drug for ameliorating male climacteric disorders comprising one kind or two or more kinds of members selected from tangshenosides, lancemaside-A, and syringin.

7. A pharmaceutical drug comprising one kind or two or more kinds of members selected from tangshenosides, lancemaside-A, and syringin.

8. A food comprising one kind or two or more kinds of members selected from tangshenosides, lancemaside-A, and syringin.

9. The pharmaceutical drug according to claim 7, further comprising one kind or two or more kinds of drugs selected from drugs having antidepressive effect, drugs having antianxiety effect, and drugs having inhibitory effect on reduction in blood DHEA-S level.

10. Use of a plant belonging to the genus Codonopsis or an extract thereof for producing a drug for inhibiting reduction in blood testosterone level.

11. Use of a plant belonging to the genus Codonopsis or an extract thereof for producing a drug for ameliorating male climacteric disorders.

12. Use of tangshenosides, lancemaside-A, and syringin for producing a drug for inhibiting reduction in blood testosterone level.

13. Use of tangshenosides, lancemaside-A, and syringin for producing a drug for ameliorating male climacteric disorders.

14. A method for inhibiting reduction in blood testosterone level, characterized by administering or ingesting a plant belonging to the genus Codonopsis or an extract thereof.

15. A method for ameliorating male climacteric disorders, characterized by administering or ingesting a plant belonging to the genus Codonopsis or an extract thereof.

16. A method for inhibiting reduction in blood testosterone level, characterized by administering or ingesting one kind or two or more kinds of members selected from tangshenosides, lancemaside-A, and syringin.

17. A method for ameliorating male climacteric disorders, characterized by administering or ingesting one kind or two or more kinds of members selected from tangshenosides, lancemaside-A, and syringin.
